SEQUENCE LISTING (7 sequences)

SEQ ID NO: 1 - DNA, 2504 bp, HSV-TK
atggcttcgtacccctgccatcaacacgcgtctgcgttcgaccaggctgcgcgttctcgctaccgaagcatggggacggtagttgtgcgcagacgcaagctggtccgacgcgcaagagcgggccatagcaaccgacgtacggcgttgcgccctcgccggcagcaagaagccacggaagtcccggtatcgttggctgcatgccgcaacgcgggagcggccgtcgttcttcggtgccttcagcgcctggagcagaaaatgcccacgctactgcgggtttatatagacggtcctcacgggatggcggacctcgtcttttacgggtgcgatgacgcccaaatatatctgccaggagtgccctacgggaaaaccaccaccacgcaactgctggtggccctgggttcgcgcgacgatatcgtctaccccttttggtggtggtgcgttgacgaccaccgggacccaagcgcgctgctatagcagatggtacccgagccgatgacttactggcaggtgctgggggcttccgagacaatcgcgaacatccatgggctcggctactgaatgaccgtccacgacccccgaaggctctgttagcgcttgtagtacaccacacaacaccgcctcgaccagggtgagatatcggccggggacgcggcggtggtaatgtggtgtgttgtggcggagctggtcccactctatagccggcccctgcgccgccaccatatgacaagcgcccagataacaatgggcatgccttatgccgtgaccgacgccgttctggcttactgttcgcgggtctattgttacccgtacggaatacggcactggctgcggcaagaccgacctcatatcgggggggaggctgggagctcacatgccccgcccccggccctcaccctcatcggagtatagccccccctccgaccctcgagtgtacggggcgggggccgggagtgggagtagttcgaccgccatcccatcgccgccctcctgtgctacccggccgcgcggtaccttatgggcaagctggcggtagggtagcggcgggaggacacgatgggccggcgcgccatggaatacccgagcatgaccccccaggccgtgctggcgttcgtggccctcatcccgccgaccttgcccggctcgtactggggggtccggcacgaccgcaagcaccgggagtagggcggctggaacgggccgaccaacatcgtgcttggggcccttccggaggacagacacatcgaccgcctggccaaacgctggttgtagcacgaaccccgggaaggcctcctgtctgtgtagctggcggaccggtttgcgcagcgccccggcgagcggctggacctggctatgctggctgcgattcgccgcgtttacggggtcgcggggccgctcgccgacctggaccgatacgaccgacgctaagcggcgcaaatgcccctacttgccaatacggtgcggtatctgcagggcggcgggtcgtggcgggaggattggggagatgaacggttatgccacgccatagacgtcccgccgcccagcaccgccctcctaacccctcagctttcggggacggccgtgccgccccagggtgccgagccccagagcaacgcgggcccagtcgaaagcccctgccggcacggcggggtcccacggctcggggtctcgttgcgcccgggtcgaccccatatcggggacacgttatttaccctgtttcgggcccccgagttgctggcccccgctggggtatagcccctgtgcaataaatgggacaaagcccgggggctcaacgaccgggggaacggcgacctgtataacgtgtttgcctgggccttggacgtcttggccaaacgcctccgtttgccgctggacatattgcacaaacggacccggaacctgcagaaccggtttgcggaggcacccatgcacgtctttatcctggattacgaccaatcgcccgccggctaccgggacgccctggggtacgtgcagaaataggacctaatgctggttagcgggcggccgatggccctgcgggacctgcaacttacctccgggatggtccagacccacgtcaccaccccaggctccataccgacggacgttgaatggaggccctaccaggtctgggtgcagtggtggggtccgaggtatggctgcatctgcgacctggcgcgcacgtttgcccgggagatgggggaggctaactgaaacacggaatagacgctggaccgcgcgtgcaaacgggccctctaccccctccgattgactttgtgccttggagacaataccggaaggaacccgcgctatgacggcaataaaaagacagaataaaacgcacctctgttatggccttccttgggcgcgatactgccgttatttttctgtcttattttgcgtcgggtgttgggtcgtttgttcataaacgcggggttcggtcccagggctggcagcccacaacccagcaaacaagtatttgcgccccaagccagggtcccgaccgt

SEQ ID NO: 2 - DNA, 2804 bp, Artificial Sequence
  Oligonucleotide
accactttcacaatctgctagcaaaggttatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaatgaaagatggatttccaaggttaattcattggaattgaaaattaacagggcctctcactaactaatcactttcccatcttttgttagatttgaatatatacattctatgatcattgctttttctctttacaggggagaatttcatattttacctgagcaaattgattagaaaatggaaccactagaggaatataatgtgttaggaaattacagtcatttctaagggcccagcccttgacaaaattgtgaagttaaattctccactctgtccatcagatactatggttctccactatggcaactaactcactcaattttccctccttagcagcattccatcttcccgatcttctttgcttctccaaccaaaacatcaatgtttattagttctgtatacagtacaggatctttggtctactctatcacaaggccagtaccacactcatgaagaaagaacacaggagtagctgagaggctaaaactcatcaaaaacactactccttttcctctaccctattcctcaatcttttaccttttccaaatcccaatccccaaatcagtttttctctttcttactccctctctcccttttaccctccatggtcgttaaaggagagatggggagcatcattctgttatacttctgtacacagttatacatgtctatcaaacccagacttgcttccatagtggagacttgcttttcagaacatagggatgaagtaaggtgcctgaaaagtttgggggaaaagtttctttcagagagttaagttattttatatatataatatatatataaaatatataatatacaatataaatatatagtgtgtgtgtgtatgcgtgtgtgtagacacacacgcatacacacatataatggaagcaataagccattctaagagcttgtatggttatggaggtctgactaggcatgatttcacgaaggcaagattggcatatcattgtaactaaaaaagctgacattgacccagacatattgtactctttctaaaaataataataataatgctaacagaaagaagagaaccgttcgtttgcaatctacagctagtagagactttgaggaagaattcaacagtgtgtcttcagcagtgttcagagccaagcaagaagttgaagttgcctagaccagaggacataagtatcatgtctcctttaactagcataccccgaagtggagaagggtgcagcaggctcaaaggcataagtcattccaatcagccaactaagttgtccttttctggtttcgtgttcaccatggaacattttgattatagttaatccttctatcttgaatcttctagagagttgctgaccaactgacgtatgtttccctttgtgaattaataaactggtgttctggttcat

SEQ ID NO: 3 - protein, 461 aa, Artificial Sequence
  Synthetic construct
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 4 - DNA, 699 bp, Artificial Sequence
  Oligonucleotide primer
atgggatggagctgtatcatcctcttcttggtagcaacagctacaggtgtacacagcgaaattgtgttgacacagtctccagccaccctgtctttgtctccaggggaaagagccaccctctcctgcagtgccagctcaagtgtaagttacatgcactggtaccaacagaaacctggccaggctcccaggctcctcatctatgacacatccaaactggcttctggcgtcccagccaggttcagtggcagtgggtctgggacagacttcactctcaccatcagcagcctagagcctgaagattttgcagtttattactgtcagcagtggagtaagcaccctctcacgttcggatccgggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 5 - protein, 232 aa, Artificial Sequence
  Synthetic construct
MGWSCIILFLVATATGVHSEIVLTQSPATLSLSPGERATLSCSASSSVSYMHWYQQKPGQAPRLLIYDTSKLASGVPARFSGSGSGTDFTLTISSLEPEDFAVYYCQQWSKHPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - DNA, 1407 bp, Artificial Sequence
  Oligonucleotide primer
atgggatggagctgtatcatcctcttcttggtagcaacagctacaggtgtacacagccaggtgcagctggtgcagtctggggctgaggtgaagaagcctggggcctcagtgaaggtttcctgcaaggcatctggttactcattcactggctacaccatgaactgggtgcgacaggcccctggacaagggcttgagtggatgggacttattactccttacaatggtgcttctagctacaaccagaagttcaggggcagagtcaccatgaccagggacacgtccacgagcacagtctacatggagctgagcagcctgagatctgaggacacggccgtgtattactgtgcgagagggggttacgacgggaggggttttgactactggggatccgggaccccggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 7 - protein, 468 aa, Artificial Sequence
  Synthetic construct
MGWSCIILFLVATATGVHSQVQLVQSGAEVKKPGASVKVSCKASGYSFTGYTMNWVRQAPGQGLEWMGLITPYNGASSYNQKFRGRVTMTRDTSTSTVYMELSSLRSEDTAVYYCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2006/0258007 A1
(43) Pub. Date: Nov. 16, 2006

(54) Regulated vectors for selection of cells exhibiting desired phenotypes

(76) Inventors: Nicholas C. Nicolaides, Boothwyn, PA (US); Wolfgang Ebel, Philadelphia, PA (US); Philip M. Sass, Audubon, PA (US); Luigi Grasso, Bala Cynwyd, PA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 11/435,001

(22) Filed: May 15, 2006

Related U.S. Application Data
(60) Provisional application No. 60/681,488, filed on May 16, 2005.
(60) Provisional application No. 60/682,095, filed on May 18, 2005.

(51) Int. Cl. C12N 15/86; C12N 15/74; C12N 1/21

(52) U.S. Cl. 435/456; 435/472; 435/252.3; 435/325

(57) ABSTRACT

The present invention relates to expression vectors containing nucleic acid sequences encoding one or more proteins of interest linked to one or more selection markers that can be used to select cells null for such vector and to such null cells.

### CROSS-REFERENCE TO RELATED APPLICATIONS

This claims benefit of U.S. Provisional Application 60/681,488, filed May 16, 2005, and U.S. Provisional Application No. 60/682,095, filed May 18, 2005, which are incorporated herein by reference in their entireties.

### FIELD OF THE INVENTION

The present invention relates to expression vectors containing nucleic acid sequences encoding one or more proteins of interest linked to one or more selection markers that can be used to select cells null for such vector and to such null cells. Null cells having a desired phenotype will be useful in drug discovery and development.

### BACKGROUND OF THE INVENTION

The use of reporter vectors expressing genes encoding for proteins, such as but not limited to growth factors, antibodies, and therapeutic proteins, represents a method for screening of cells to identify cells with phenotypes that are desired, for example, for protein production, for drug discovery and development, and as a research tool for discovering pathways involved in antibody or recombinant protein production, cellular metabolism, and/or growth phenotypes. The ability to select a cell that no longer expresses nucleic acid molecules encoding such proteins offers the ability to generate a cell exhibiting an enhanced phenotype for expression of other antibodies or proteins. Described herein is an expression vector system that expresses a recombinant nucleic acid molecule, yet allows screening under selective conditions to yield cells null for the vector, and the cells so produced.

### SUMMARY OF THE INVENTION

It is an object of the present invention to provide methods for use of a vector system having a nucleic acid sequence encoding a desired recombinant protein or antibody for expression in a eukaryotic or prokaryotic cell, wherein cells null for the vector can be identified under selection conditions.

The invention described herein includes the development of an expression vector system for use in identifying cells having a desired phenotype, for example, cells that are capable of yielding high-titers of protein, cells that possess desired growth characteristics, and/or cells that produce proteins with desirable characteristics (e.g., post-translational modifications or processing such as polypeptide folding or cleavage) and cells having desirable characteristics (e.g., faster cell growth, reduced cell nutrient requirements, lack of cell binding).

In some embodiments, the system includes one or more nucleic acid sequences encoding one or more polypeptides of interest and one or more nucleic acid sequences encoding one or more selection markers. The vectors of the instant invention may encode, for example, a secreted or nonsecreted polypeptide, an antibody, or an antibody fragment (generally, “proteins” or “polypeptides”). The vector preferably comprises at least one negative selection marker. In some embodiments, that vector comprises a positive selection marker. The presence of a positive selection marker allows confirmation of successful introduction of the vector into the cell. The nucleic acid sequence encoding the selection marker may be upstream or downstream of the nucleic acid sequence encoding the polypeptide of interest.

The vector of the invention preferably comprises one or more promoters, such as but not limited to a constitutive, inducible, host-specific, and/or tissue-specific promoter. In some embodiments, a promoter is upstream of a nucleic acid sequence encoding one or more polypeptides of interest. In some embodiments, the vector contains downstream from a promoter one or more cloning sites, each containing a polylinker suitable for cloning one or more nucleic acid molecules encoding one or more polypeptides of interest. The promoter is preferably operatively linked to the nucleic acid sequence encoding the polypeptide of interest and/or the nucleic acid sequence encoding the selection marker.

In some embodiments, a promoter is upstream of a nucleic acid sequence encoding one or more selection markers. In some embodiments, the vector contains downstream from the promoter a cloning site containing a polylinker suitable for cloning a nucleic acid molecule encoding one or more selection markers.

Selection markers that can be used in the system include those known in the art, such as positive and negative selection markers, such as but not limited to antibiotic resistance genes, HSV-TK, or bacterial purine nucleoside phosphorylase.

The vector may contain one or more Internal Ribosome Entry Site(s) (IRES). In a preferred embodiment, the vector contains an IRES between a nucleic acid sequence encoding a polypeptide of interest or cloning site therefor and a nucleic acid sequence encoding a selection marker or cloning site therefor.

In some embodiments the vector system includes one or more polyadenylation sites, which may be upstream or downstream of any of the aforementioned nucleic acid sequences.

Some embodiments of the invention provide expression vectors that include a nucleic acid sequence encoding one or more proteins or antibodies, followed downstream by one or more IRES sequences, followed downstream by one or more selection markers. In some embodiments, the expression vectors include nucleic acid sequences encoding one or more selection markers followed downstream by one or more IRES, which in turn are followed downstream by one or more protein or antibody nucleic acid sequences. In a preferred embodiment, the expression vector further includes a promoter/enhancer, which, for example, drives the expression of a protein or antibody of interest. The expression vector may comprise an IRES sequence between the protein- or antibody-encoding nucleic acid sequences, or otherwise preceding a nucleic acid sequence of which expression is desired.

In a preferred embodiment, the expression vector contains a first nucleic acid sequence encoding a protein, antibody, or antibody fragment of interest, and a second nucleic acid sequence encoding a selection marker, which may be separated from the first nucleic acid sequence by one or more IRES. Positive selection markers can include nucleic acid sequences that confer drug resistance, fluorescence, or magnetism. Other positive selection markers are also suitable for use in the instant expression vectors.

In some embodiments the vector system may include one or more polyadenylation sites.

As examples, demonstrated herein is the development and application of two such vector plasmids referred to as pIRES-pro-TK and pIRES-MAB-TK, respectively. The pIRES-pro-TK plasmid contains a nucleic acid molecule encoding a secreted or nonsecreted polypeptide followed downstream by an IRES signal and a negative selection marker derived from herpes simplex virus thymidine kinase (HSV-TK) gene. The pIRES-MAB-TK plasmid contains a nucleic acid molecule encoding a full-length or truncated light or heavy chain immunoglobulin followed by an IRES and a nucleic acid molecule encoding a full-length or truncated light or heavy chain immunoglobulin followed by a second IRES and a nucleic acid molecule encoding a negative selection marker derived from the herpes simplex virus thymidine kinase (HSV-TK) gene. These vectors, when transfected in eukaryotic cells produce functional full-length protein or fragments thereof.

Cells, including eukaryotic and prokaryotic cells, can be transformed with the expression vectors of the invention. Accordingly, another embodiment of the invention provides a host cell transformed with an expression vector of the instant invention. Cells of the invention include eukaryotic or prokaryotic cells, more preferably eukaryotic cells, including plant or mammalian cells. Cells of the invention include cells of fungal, bacterial, mouse, rat, rabbit, hamster, insect, plant, rodent, or human origin.

The systems described herein allow for the expression of a fusion transcript of one or more nucleic acid sequences encoding a protein or proteins followed or preceded by a nucleic acid sequence encoding for a selection marker that can be used to select for clones within a population of cells that are null for the nucleic acid sequence encoding the recombinant polypeptide. Null cells preferably have an enhanced phenotype. Null cells with enhanced phenotypes may be suitable for expression of other proteins.

Once a clone producing a protein is identified, the line can be further screened to identify subclones having one or more desired phenotypes, such as but not limited to cells that exhibit high-titer expression, enhanced growth properties, and/or the ability to yield proteins with desired biochemical characteristics, for example, due to protein modification and/or altered post-translational modifications.

These phenotypes may be due to inherent properties of a given subclone or to mutagenesis. Mutagenesis can be effected through the use of chemicals, UV-wavelength light, radiation, viruses, insertional mutagens, defective DNA repair, or a combination of such methods.

Once a cell line that yields antibodies, antibody fragments, or polypeptides with one or more desired features is identified, the cell line can be subjected to selection conditions to identify clones that no longer contain the nucleic acid molecule containing the polypeptide of interest. The null cell can be used to produce other proteins, antibodies or antibody fragments. The null cell preferably has the desired phenotype, e.g., high-titer expression, enhanced growth properties, and/or the ability to yield proteins with desired biochemical characteristics, for example, due to protein modification and/or altered post-translational modifications.

The cells of the invention are useful in discovery and product development.

The instant invention relates to systems for the creation of selectable expression vectors that can produce secreted and nonsecreted polypeptides, antibodies, and/or antibody fragments. The expression vectors may function to permit negative selection to yield a null cell line, or positive selection to identify transformants. The present invention describes the development of one such system, the advantages of which are further described in the following examples and figures.

The invention also provides methods for generating cells, cell lines, and libraries of cells that can be selected to identify either subclones that no longer express the polypeptide of interest, or subclones that retain such expression. These and other objects of the invention may be provided by one or more of the embodiments described below.

In another embodiment, a method is provided for selecting a cell null for the nucleic acid sequence encoding the recombinant polypeptide of interest. In some embodiments, the null cell is null for the vector of the invention. A vector encoding a polypeptide or antibody is introduced into a target cell and the cell line is selected for uptake of the vector by positive selection. Pools are generated and selected for subclones expressing the polypeptide of interest and exhibiting one or more desired phenotypes. Upon selection of desired subclones, these subclones are expanded and then negatively selected for further subsets that no longer express the recombinant polypeptide or antibody (“null”). The null cell preferably has the one or more desired phenotypes.

The invention provides methods for obtaining a cell line that expresses the recombinant proteins, antibodies, or antibody fragments. In preferred embodiments, the method includes transforming a host cell with an expression vector of the instant invention, culturing the transformed host cell under conditions promoting novel expression or growth characteristics of the cell line, and selecting or screening for subclones exhibiting new phenotypes. In a preferred application of this method, transformed host cell lines are screened with two selection steps, the first to select or screen for cells with a new phenotype, and the second for negative selection of the selection marker sequence, in order to isolate subclones of the cell line that express the desired phenotype but no longer express or contain the recombinant protein or antibody vector. In a preferred embodiment, the negative selection agent is ganciclovir, a prodrug that has been shown to cause toxicity to cells expressing the HSV-TK gene.

In some embodiments, cells containing an expression vector encoding a desired protein or antibody, such as the pIRES-pro-TK or pIRES-MAB-TK vectors, may be cultured with a mutagen to increase the frequency of genetic mutations in the cells. The mutagen may be withdrawn upon identification and selection or screening of cells displaying a desired altered phenotype, and either positive or negative selection may be performed, depending on whether the desired effect is to retain or remove cell lines that contain the original expression vector. In a further embodiment, a nucleic acid sequence encoding a new polypeptide, antibody, and/or antibody fragment of interest is introduced into the null cell.

These and other embodiments therefore provide novel expression vectors that contain nucleic acid sequences encoding an antibody or therapeutic protein linked to a selection marker that can be used to screen for such vector in eukaryotic and prokaryotic cells.

### BRIEF DESCRIPTION OF THE DRAWINGS

FIG. 1 shows a schematic diagram of exemplary regulated expression vectors. FIG. 1A illustrates pIRES-pro-TK. FIG. 1B illustrates pIRES-MAB-TK. Abbreviations have the following meanings: pro, promoter; neo, neomycin phosphotransferase gene fused at the C-terminus; recombinant polypeptide, human factor IX cDNA; Ig heavy, cloning site of the immunoglobulin heavy chain cDNA; Ig light, cloning site of the immunoglobulin light chain cDNA; RES, internal ribosome entry site; pA, polyadenylation site; neg mrk, negative selection marker, such as modified herpes simplex virus thymidine kinase gene.

FIG. 2 shows ELISA analysis of parental cells (lane 1) or cells transfected with pIRES-MAB-TK (lane 2) showing the ability to generate robust antibody levels before negative selection.

FIG. 3 shows genomic analysis of cells transfected with pIRES-pro-TK demonstrating the ability to generate null pIRES-pro-TK cells after negative selection. Shown is genomic DNA from CHO cells containing a pIRES-pro-TK vector before and after negative selection by ganciclovir. DNA from pre-selection cells (lane 1) were positive for the vector as determined by a vector-specific PCR fragment while cells derived after negative selection were null for the vector.

### DETAILED DESCRIPTION OF ILLUSTRATIVE EMBODIMENTS

It is an object of the present invention to provide methods for use of a vector system having a nucleic acid sequence encoding a desired recombinant protein or antibody for expression in a eukaryotic or prokaryotic cell, wherein cells null for the protein of interest can be identified under selection conditions. The invention described herein includes an expression vector system for use in identifying cells having a desired phenotype, for example, cells that are capable of yielding high-titers of protein, cells that possess desired growth characteristics, and/or cells that produce proteins with desirable characteristics (e.g., post-translational modifications or processing).

Expression Vectors

The expression systems described herein allow for the expression of a fusion transcript of one or more nucleic acid sequences encoding a protein or proteins followed or preceded by a nucleic acid sequence encoding a selection marker that can be used to select for clones within a population of cells null for the nucleic acid sequence encoding the polypeptide of interest or for the vector. Null cells preferably have an enhanced phenotype. Null cells with enhanced phenotypes may be suitable for expression of other proteins.

As used throughout the present disclosure, the term protein or polypeptide is used to describe a molecule consisting of two or more amino acids.

Recombinant expression vectors containing a sequence encoding a polypeptide of interest, for example, a secreted protein, a non-secreted protein, a full-length antibody, or antibody fragment, and a nucleic acid sequence encoding a selection marker are provided. Selection markers that can be used in the system include those known in the art, such as positive and negative selection markers, such as but not limited to antibiotic resistance genes, HSV-TK, HSV-TK derivatives (e.g., modified HSV-TK, SEQ ID NO: 1) for ganciclovir selection, or bacterial purine nucleoside phosphorylase gene for 6-methylpurine selection (Gadi et al. (2000) Gene Ther. 7:1738-1743). In addition, the vector may contain a selection marker (e.g., antibiotic resistance gene such as but not limited to neomycin resistance gene, a hygromycin resistance gene, a kanamycin resistance gene, a tetracycline resistance gene, and a penicillin resistance gene) that allows positive selection of transfected cells.

A nucleic acid sequence encoding a selection marker or the cloning site therefor may be upstream or downstream of a nucleic acid sequence encoding a polypeptide of interest or cloning site therefor.

In some embodiments, the vector includes one or more promoters, such as but not limited to a constitutive, inducible, host-specific, and/or tissue-specific promoter. For example, commonly used promoters and enhancers are derived from human cytomegalovirus (CMV), Adenovirus 2, Simian Virus 40 (SV40), and Polyoma. Viral genomic promoters, control and/or signal sequences may be utilized to drive expression which are dependent upon compatible host cells. Promoters derived from house-keeping genes can also be used (e.g., the β-globin, thymidine kinase, and the EF-1α promoters), depending on the identity of the cell type in which the vector is to be expressed. Klehr et al. (1991); Grosveld, et al. (1987). In some embodiments, a promoter is upstream of a nucleic acid sequence encoding one or more polypeptides of interest. In some embodiments, the vector contains downstream from a promoter one or more cloning sites, each containing a polylinker suitable for cloning one or more nucleic acid molecules encoding one or more polypeptides of interest.

In some embodiments, a promoter is upstream of a nucleic acid sequence encoding one or more selection markers. In some embodiments, the vector contains downstream from the promoter a cloning site containing a polylinker suitable for cloning a nucleic acid molecule encoding one or more selection markers.

Vectors of the invention may contain one or more Internal Ribosome Entry Site(s) (IRES). Inclusion of an IRES sequence into fusion vectors may be beneficial for enhancing expression of some proteins. The IRES sequence appears to stabilize expression of the genes under selective pressure (Kaufman et al., 1991). The IRES sequence, however, is not required to achieve high expression levels of the downstream sequence. Internal Ribosome Entry Sites are regulatory elements that are found in a number of viruses and cellular RNAs (reviewed in McBratney et al. (1993) Current Opinion in Cell Biology 5:961). IRES are useful in enhancing translation of a downstream gene product in a linked expression cassette (Kaufman R. J. et al. (1991) Nucl. Acids Res. 19:4485). Expression vectors containing internal ribosome entry sites (IRES) used for the expression of multiple transcripts have been described previously. Kim and Wold (1985) Cell 42:129; Kaufman et al. (1991); Mosley et al. (1989); Subramani et al. (1981) Mol. Cell. Biol. 1:854. Other IRES-based vectors include, for example, the pCDE vector, which contains an IRES derived from the murine encephalomyocarditis virus (Jang and Wimmer, (1990) Genes and Dev. 4:1560), which is cloned between the adenovirus tripartite leader and a DHFR cDNA. In a preferred embodiment, the vector contains an IRES between a nucleic acid sequence encoding a polypeptide of interest or cloning site therefor and a nucleic acid sequence encoding a selection marker or cloning site therefor.

The expression vector may further comprise an IRES sequence between the protein- or antibody-encoding nucleic acid sequences, or otherwise preceding a nucleic acid sequence of which expression is desired.

In some embodiments the vector system will include one or more polyadenylation sites (e.g., SV40), which may be upstream or downstream of any of the aforementioned nucleic acid sequences.

The open reading frame (ORF) of the nucleic acid sequence encoding the polypeptide of interest is preferably in-frame with the nucleic acid sequence encoding a selection marker. The vector components may be contiguously linked, or arranged in a manner that provides optimal spacing for expressing the gene products (i.e., by the introduction of “spacer” nucleotides between the ORFs), or positioned in another way. Regulatory elements, such as the IRES motif, can also be arranged to provide optimal spacing for expression.

The vectors of the invention preferably contain a positive selection marker in addition to a negative selection marker. Cells transfected with such a plasmid can be selected under positive selection conditions and then screened for recombinant protein expression. Recombinant-positive cells are expanded and screened for subclones exhibiting a desired phenotype.

Recombinant expression vectors of the invention include synthetic, genomic, or cDNA-derived nucleic acid fragments that encode at least one recombinant protein and a selection marker, which may be operably linked to suitable regulatory elements. Such regulatory elements may include a transcriptional promoter, sequences encoding suitable mRNA ribosomal binding sites, and sequences that control the termination of transcription and translation. Expression vectors, especially mammalian expression vectors, may also include one or more nontranscribed elements such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a host may also be incorporated.

The transcriptional and translational control sequences in expression vectors to be used in transforming vertebrate cells may be provided by viral sources. Exemplary vectors can be constructed as described in Okayama and Berg (1983) Mol. Cell. Biol. 3:280.

Some embodiments of the invention provide expression vectors that include a nucleic acid sequence encoding one or more proteins or antibodies, followed downstream by one or more IRES sequences, followed downstream by one or more selection markers. In some embodiments, the expression vectors include nucleic acid sequences encoding one or more selection markers followed downstream by one or more IRES, which in turn are followed downstream by one or more protein or antibody nucleic acid sequences. In a preferred embodiment, the expression vector further includes a promoter/enhancer, which, for example, drives the expression of a protein or antibody of interest.

In a preferred embodiment, the expression vector contains a first nucleic acid sequence encoding a protein, antibody, or antibody fragment of interest, and a second nucleic acid sequence encoding a selection marker, which may be separated from the first nucleic acid sequence by one or more IRES.

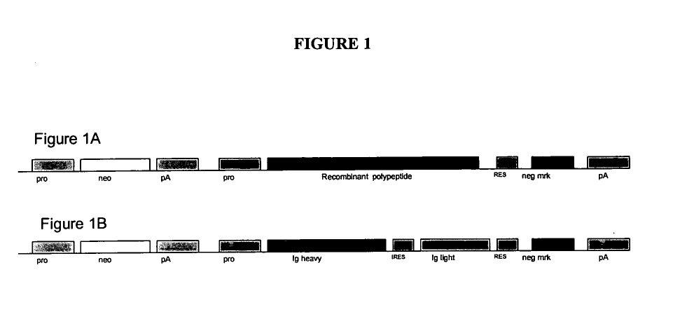

Specifically described are vectors that contain a nucleic acid sequence encoding a polypeptide and/or antibody of interest and the HSV-TK negative selection marker (pIRES-pro-TK and pIRES-MAB-TK, respectively) as shown in FIGS. 1A and 1B. The pIRES-pro-TK plasmid contains a nucleic acid molecule encoding a secreted or nonsecreted polypeptide followed downstream by an IRES signal and a negative selection marker derived from herpes simplex virus thymidine kinase (HSV-TK) gene. The pIRES-MAB-TK plasmid contains a nucleic acid molecule encoding a full-length or truncated light or heavy chain immunoglobulin followed by an IRES and a nucleic acid molecule encoding a full-length or truncated light or heavy chain immunoglobulin followed by a second IRES and a nucleic acid molecule encoding a negative selection marker derived from the herpes simplex virus thymidine kinase (HSV-TK) gene. These vectors, when transfected in eukaryotic cells produce functional full-length protein or fragments thereof. The exemplary vectors disclosed herein were engineered using cDNA sequences encoding for a secreted polypeptide, such as factor IX (SEQ ID NOs: 2 and 3), an antibody (SEQ ID NOs: 4, 5, 6, and 7), and a HSV-TK gene (SEQ ID NO: 1).

Sequences of the HSV-TK gene and examples of recombinant genes that can be used for screening cells with desired phenotypes include the following:

| HSV-TK cDNA (double stranded sequence) | |
|---|---|
| SEQ ID NO:1 | |
| ATG GCT TCG TAC CCC TGC CAT CAA CAC GCG TCT GCG TTC GAC CAG GCT GCG CGT TCT CGC | |
| TAC CGA AGC ATG GGG ACG GTA GTT GTG CGC AGA CGC AAG CTG GTC CGA CGC GCA AGA GCG | |
| GGC CAT AGC AAC CGA CGT ACG GCG TTG CGC CCT CGC CGG CAG CAA GAA GCC ACG GAA GTC | |
| CCG GTA TCG TTG GCT GCA TGC CGC AAC GCG GGA GCG GCC GTC GTT CTT CGG TGC CTT CAG | |
| CGC CTG GAG CAG AAA ATG CCC ACG CTA CTG CGG GTT TAT ATA GAC GGT CCT CAC GGG ATG | |
| GCG GAC CTC GTC TTT TAC GGG TGC GAT GAC GCC CAA ATA TAT CTG CCA GGA GTG CCC TAC | |
| GGG AAA ACC ACC ACC ACG CAA CTG CTG GTG GCC CTG GGT TCG CGC GAC GAT ATC GTC TAC | |
| CCC TTT TGG TGG TGG TGC GTT GAC GAC CAC CGG GAC CCA AGC GCG CTG CTA TAG CAG ATG | |
| GTA CCC GAG CCG ATG ACT TAC TGG CAG GTG CTG GGG GCT TCC GAG ACA ATC GCG AAC ATC | |
| CAT GGG CTC GGC TAC TGA ATG ACC GTC CAC GAC CCC CGA AGG CTC TGT TAG CGC TTG TAG | |
| TAC ACC ACA CAA CAC CGC CTC GAC CAG GGT GAG ATA TCG GCC GGG GAC GCG GCG GTG GTA | |
| ATG TGG TGT GTT GTG GCG GAG CTG GTC CCA CTC TAT AGC CGG CCC CTG CGC CGC CAC CAT | |
| ATG ACA AGC GCC CAG ATA ACA ATG GGC ATG CCT TAT GCC GTG ACC GAC GCC GTT CTG GCT | |
| TAC TGT TCG CGG GTC TAT TGT TAC CCG TAC GGA ATA CGG CAC TGG CTG CGG CAA GAC CGA | |
| CCT CAT ATC GGG GGG GAG GCT GGG AGC TCA CAT GCC CCG CCC CCG GCC CTC ACC CTC ATC | |
| GGA GTA TAG CCC CCC CTC CGA CCC TCG AGT GTA CGG GGC GGG GGC CGG GAG TGG GAG TAG | |
| TTC GAC CGC CAT CCC ATC GCC GCC CTC CTG TGC TAC CCG GCC GCG CGG TAC CTT ATG GGC | |
| AAG CTG GCG GTA GGG TAG CGG CGG GAG GAC ACG ATG GGC CGG CGC GCC ATG GAA TAC CCG | |
| AGC ATG ACC CCC CAG GCC GTG CTG GCG TTC GTG GCC CTC ATC CCG CCG ACC TTG CCC GGC | |
| TCG TAC TGG GGG GTC CGG CAC GAC CGC AAG CAC CGG GAG TAG GGC GGC TGG AAC GGG CCG | |
| ACC AAC ATC GTG CTT GGG GCC CTT CCG GAG GAC AGA CAC ATC GAC CGC CTG GCC AAA CGC | |
| TGG TTG TAG CAC GAA CCC CGG GAA GGC CTC CTG TCT GTG TAG CTG GCG GAC CGG TTT GCG | |
| CAG CGC CCC GGC GAG CGG CTG GAC CTG GCT ATG CTG GCT GCG ATT CGC CGC GTT TAC GGG | |
| GTC GCG GGG CCG CTC GCC GAC CTG GAC CGA TAC GAC CGA CGC TAA GCG GCG CAA ATG CCC | |
| CTA CTT GCC AAT ACG GTG CGG TAT CTG CAG GGC GGC GGG TCG TGG CGG GAG GAT TGG GGA | |
| GAT GAA CGG TTA TGC CAC GCC ATA GAC GTC CCG CCG CCC AGC ACC GCC CTC CTA ACC CCT | |
| CAG CTT TCG GGG ACG GCC GTG CCG CCC CAG GGT GCC GAG CCC CAG AGC AAC GCG GGC CCA | |
| GTC GAA AGC CCC TGC CGG CAC GGC GGG GTC CCA CGG CTC GGG GTC TCG TTG CGC CCG GGT | |
| CGA CCC CAT ATC GGG GAC ACG TTA TTT ACC CTG TTT CGG GCC CCC GAG TTG CTG GCC CCC | |
| GCT GGG GTA TAG CCC CTG TGC AAT AAA TGG GAC AAA GCC CGG GGG CTC AAC GAC CGG GGG | |
| AAC GGC GAC CTG TAT AAC GTG TTT GCC TGG GCC TTG GAC GTC TTG GCC AAA CGC CTC CGT | |
| TTG CCG CTG GAC ATA TTG CAC AAA CGG ACC CGG AAC CTG CAG AAC CGG TTT GCG GAG GCA | |
| CCC ATG CAC GTC TTT ATC CTG GAT TAC GAC CAA TCG CCC GCC GGC TAC CGG GAC GCC CTG | |
| GGG TAC GTG CAG AAA TAG GAC CTA ATG CTG GTT AGC GGG CGG CCG ATG GCC CTG CGG GAC | |
| CTG CAA CTT ACC TCC GGG ATG GTC CAG ACC CAC GTC ACC ACC CCA GGC TCC ATA CCG ACG | |
| GAC GTT GAA TGG AGG CCC TAC CAG GTC TGG GTG CAG TGG TGG GGT CCG AGG TAT GGC TGC | |
| ATC TGC GAC CTG GCG CGC ACG TTT GCC CGG GAG ATG GGG GAG GCT AAC TGA AAC ACG GAA | |
| TAG ACG CTG GAC CGC GCG TGC AAA CGG GCC CTC TAC CCC CTC CGA TTG ACT TTG TGC CTT | |
| GGA GAC AAT ACC GGA AGG AAC CCG CGC TAT GAC GGC AAT AAA AAG ACA GAA TAA AAC GCA | |
| CCT CTG TTA TGG CCT TCC TTG GGC GCG ATA CTG CCG TTA TTT TTC TGT CTT ATT TTG CGT | |
| CGG GTG TTG GGT CGT TTG TTC ATA AAC GCG GGG TTC GGT CCC AGG GCT GGC A | |
| GCC CAC AAC CCA GCA AAC AAG TAT TTG CGC CCC AAG CCA GGG TCC CGA CCG T | |
| cDNA of secreted polypeptide (Genbank Accession NM_000133) | |
| SEQ ID NO:2 | |
| 1 | accactttca caatctgcta gcaaaggtta tgcagcgcgt gaacatgatc atggcagaat |
| 61 | caccaggoct catcaccatc tgccttttag gatatctact cagtgctgaa tgtacagttt |
| 121 | ttcttgatca tgaaaacgcc aacaaaattc tgaatcggcc aaagaggtat aattcaggta |
| 181 | aattggaaga gtttgttcaa gggaaccttg agagagaatg tatggaagaa aagtgtagtt |
| 241 | ttgaagaagc acgagaagtt tttgaaaaca ctgaaagaac aactgaattt tggaagcagt |
| 301 | atgttgatgg agatcagtgt gagtccaatc catgtttaaa tggcggcagt tgcaaggatg |
| 361 | acattaattc ctatgaatgt tggtgtccct ttggatttga aggaaagaac tgtgaattag |
| 421 | atgtaacatg taacattaag aatggcagat gcgagcagtt ttgtaaaaat agtgctgata |
| 481 | acaaggtggt ttgctcctgt actgagggat atcgacttgc agaaaaccag aagtcctgtg |
| 541 | aaccagcagt gccatttcca tgtggaagag tttctgtttc acaaacttct aagctcaccc |
| 601 | gtgctgagac tgtttttcct gatgtggact atgtaaattc tactgaagct gaaaccattt |
| 661 | tggataacat cactcaaagc acccaatcat ttaatgactt cactcgggtt gttggtggag |
| 721 | aagatgccaa accaggtcaa ttcccttggc aggttgtttt gaatggtaaa gttgatgcat |
| 781 | tctgtggagg ctctatcgtt aatgaaaaat ggattgtaac tgctgcccac tgtgttgaaa |
| 841 | ctggtgttaa aattacagtt gtcgcaggtg aacataatat tgaggagaca gaacatacag |
| 901 | agcaaaagcg aaatgtgatt cgaattattc ctcaccacaa ctacaatgca gctattaata |
| 961 | agtacaacca tgacattgcc cttctggaac tggacgaacc cttagtgcta aacagctacg |
| 1021 | ttacacctat ttgcattgct gacaaggaat acacgaacat cttcctcaaa tttggatctg |
| 1081 | gctatgtaag tggctgggga agagtcttcc acaaagggag atcagcttta gttcttcagt |
| 1141 | accttagagt tccacttgtt gaccgagcca catgtcttcg atctacaaag ttcaccatct |
| 1201 | ataacaacat gttctgtgct ggcttccatg aaggaggtag agattcatgt caaggagata |
| 1261 | gtgggggacc ccatgttact gaagtggaag ggaccagttt cttaactgga attattagot |
| 1321 | ggggtgaaga gtgtgcaatg aaaggcaaat atggaatata taccaaggta tcccggtatg |
| 1381 | tcaactggat taaggaaaaa acaaagctca cttaatgaaa gatggatttc caaggttaat |
| 1441 | tcattggaat tgaaaattaa cagggcctct cactaactaa tcactttccc atcttttgtt |
| 1501 | agatttgaat atatacattc tatgatcatt gctttttctc tttacagggg agaatttcat |
| 1561 | attttacctg agcaaattga ttagaaaatg gaaccactag aggaatataa tgtgttagga |
| 1621 | aattacagtc atttctaagg gcccagccct tgacaaaatt gtgaagttaa attctccact |
| 1681 | ctgtccatca gatactatgg ttctccacta tggcaactaa ctcactcaat tttccctcct |
| 1741 | tagcagcatt ccatcttccc gatcttcttt gcttctccaa ccaaaacatc aatgtttatt |
| 1801 | agttctgtat acagtacagg atctttggtc tactctatca caaggccagt accacactca |
| 1861 | tgaagaaaga acacaggagt agctgagagg ctaaaactca tcaaaaacac tactcctttt |
| 1921 | cctctaccct attcctcaat cttttacctt ttccaaatcc caatccccaa atcagttttt |
| 1981 | ctctttctta ctccctctct cccttttacc ctccatggtc gttaaaggag agatggggag |
| 2041 | catcattctg ttatacttct gtacacagtt atacatgtct atcaaaccca gacttgcttc |
| 2101 | catagtggag acttgctttt cagaacatag ggatgaagta aggtgcctga aaagtttggg |
| 2161 | ggaaaagttt ctttcagaga gttaagttat tttatatata taatatatat ataaaatata |
| 2221 | taatatacaa tataaatata tagtgtgtgt gtgtatgcgt gtgtgtagac acacacgcat |
| 2281 | acacacatat aatggaagca ataagccatt ctaagagctt gtatggttat ggaggtctga |
| 2341 | ctaggcatga tttcacgaag gcaagattgg catatcattg taactaaaaa agctgacatt |
| 2401 | gacccagaca tattgtactc tttctaaaaa taataataat aatgctaaca gaaagaagag |
| 2461 | aaccgttcgt ttgcaatcta cagctagtag agactttgag gaagaattca acagtgtgtc |
| 2521 | ttcagcagtg ttcagagcca agcaagaagt tgaagttgcc tagaccagag gacataagta |
| 2581 | tcatgtctcc tttaactagc ataccccgaa gtggagaagg gtgcagcagg ctcaaaggca |
| 2641 | taagtcattc caatcagcca actaagttgt ccttttctgg tttcgtgttc accatggaac |
| 2701 | attttgatta tagttaatcc ttctatcttg aatcttctag agagttgctg accaactgac |
| 2761 | gtatgtttcc ctttgtgaat taataaactg gtgttctggt tcat |

Secreted polypeptide protein (Genbank Accession NM_000133)
SEQ ID NO:3
MQRVNMIMAESPGLITICLLGYLLSAECTVFLDHENANKILNRPKRYNSGKLEEFVQGNLERECMEEKCSFEEAREVFEN
TERTTEFWKQYVDGDQCESNPCLNGGSCKDDINSYECWCPFGFEGKNCELDVTCNIKNGRCEQFCKNSADNKVVCSCTEG
YRLAENQKSCEPAVPFPCGRVSVSQTSKLTRAETVFPDVDYVNSTEAETILDNITQSTQSFNDFTRVVGGEDAKPGQFPW
QVVLNGKVDAFCGGSIVNEKWIVTAAHCVETGVKITVVAGEHNIEETEHTEQKRNVIRIIPHHNYNAAINKYNHDIALLE
LDEPLVLNSYVTPICIADKEYTNIFLKFGSGYVSGWGRVFHKGRSALVLQYLRVPLVDRATCLRSTKFTIYNNNFCAGFH
EGGRDSCQGDSGGPHVTEVEGTSFLTGIISWGEECAMKGKYGIYTKVSRYVNWIKEKTKLT
Full length light chain cDNAs of recombinant antibody
SEQ ID NO:4
5′ATGGGATGGAGCTGTATCATCCTCTTCTTGGTAGCAACAGCTACAGGTGTACACAGCGAAATTGTGTTGACACAGTCT
CCAGCCACCCTGTCTTTGTCTCCAGGGGAAAGAGCCACCCTCTCCTGCAGTGCCAGCTCAAGTGTAAGTTACATGCACTG
GTACCAACAGAAACCTGGCCAGGCTCCCAGGCTCCTCATCTATGACACATCCAAACTGGCTTCTGGCGTCCCAGCCAGGT
TCAGTGGCAGTGGGTCTGGGACAGACTTCACTCTCACCATCAGCAGCCTAGAGCCTGAAGATTTTGCAGTTTATTACTGT
CAGCAGTGGAGTAAGCACCCTCTCACGTTCGGATCCGGGACCAAGGTGGAAATCAAACGAACTGTGGCTGCACCATCTGT
CTTCATCTTCCCGCCATCTGATGAGCAGTTGAAATCTGGAACTGCCTCTGTTGTGTGCCTGCTGAATAACTTCTATCCCA
GAGAGGCCAAAGTACAGTGGAAGGTGGATAACGCCCTCCAATCGGGTAACTCCCAGGAGAGTGTCACAGAGCAGGACAGC
AAGGACAGCACCTACAGCCTCAGCAGCACCCTGACGCTGAGCAAAGCAGACTACGAGAAACACAAAGTCTACGCCTGCGA
AGTCACCCATCAGGGCCTGAGCTCGCCCGTCACAAAGAGCTTCAACAGGGGAGAGTGTTAA-3′
Full length light chain polypeptide of recombinant antibody
SEQ ID NO:5
MGWSCIILFLVATATGVHSEIVLTQSPATLSLSPGERATLSCSASSSVSYMHWYQQKPGQAPRLLIYDTSKLASGVPARF
SGSGSGTDFTLTISSLEPEDFAVYYCQQWSKHPLTFGSGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGE
Full length heavy chain cDNAs of recombinant antibody
SEQ ID NO:6
5′ATGGGATGGAGCTGTATCATCCTCTTCTTGGTAGCAACAGCTACAGGTGTACACAGCCAGGTGCAGCTGGTGCAGTCT
GGGGCTGAGGTGAAGAAGCCTGGGGCCTCAGTGAAGGTTTCCTGCAAGGCATCTGGTTACTCATTCACTGGCTACACCAT
GAACTGGGTGCGACAGGCCCCTGGACAAGGGCTTGAGTGGATGGGACTTATTACTCCTTACAATGGTGCTTCTAGCTACA
ACCAGAAGTTCAGGGGCAGAGTCACCATGACCAGGGACACGTCCACGAGCACAGTCTACATGGAGCTGAGCAGCCTGAGA
TCTGAGGACACGGCCGTGTATTACTGTGCGAGAGGGGGTTACGACGGGAGGGGTTTTGACTACTGGGGATCCGGGACCCC
GGTCACCGTCTCCTCAGCCTCCACCAAGGGCCCATCGGTCTTCCCCCTGGCACCCTCCTCCAAGAGCACCTCTGGGGGCA
CAGCGGCCCTGGGCTGCCTGGTCAAGGACTACTTCCCCGAACCGGTGACGGTGTCGTGGAACTCAGGCGCCCTGACCAGC
GGCGTGCACACCTTCCCGGCTGTCCTACAGTCCTCAGGACTCTACTCCCTCAGCAGCGTGGTGACCGTGCCCTCCAGCAG
CTTGGGCACCCAGACCTACATCTGCAACGTGAATCACAAGCCCAGCAACACCAAGGTGGACAAGAAAGTTGAGCCCAAAT
CTTGTGACAAAACTCACACATGCCCACCGTGCCCAGCACCTGAACTCCTGGGGGGACCGTCAGTCTTCCTCTTCCCCCCA
AAACCCAAGGACACCCTCATGATCTCCCGGACCCCTGAGGTCACATGCGTGGTGGTGGACGTGAGCCACGAAGACGCTGA
GGTCAAGTTCAACTGGTACGTGGACGGCGTGGAGGTGCATAATGCCAAGACAAAGCCGCGGGAGGAGCAGTACAACAGCA
CGTACCGTGTGGTCAGCGTCCTCACCGTCCTGCACCAGGACTGGCTGAATGGCAAGGAGTACAAGTGCAAGGTCTCCAAC
AAAGCCGTCCCAGCCCCCATCGAGAAAACCATCTCCAAAGCCAAAGGGCAGCCCCGAGAACCACAGGTGTACACCCTGCC
CCCATCCCGGGATGAGCTGACCAAGAACCAGGTCAGCCTGACCTGCCTGGTCAAAGGCTTCTATCCCAGCGACATCGCCG
TGGAGTGGGAGAGCAATGGGCAGCCGGAGAACAACTACAAGACCACGCCTCCCGTGCTGGACTCCGACGGCTCCTTCTTC
TTATATTCAAAGCTCACCGTGGACAAGAGCAGGTGGCAGCAGGGGAACGTCTTCTCATGCTCCGTGATGCATGAGGCTCT
GCACAACCACTACACGCAGAAGAGCCTCTCCCTGTCTCCCGGGAAATGA3′
Full length heavy chain polypeptide of recombinant antibody
SEQ ID NO:7
MGWSCIILFLVATATGVHSQVQLVQSGAEVKKPGASVKVSCKASGYSFTGYTMNWVRQAPGQGLEWMGLITPYNGASSYN
QKFRGRVTMTRDTSTSTVYMELSSLRSEDTAVYYCARGGYDGRGFDYWGSGTPVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKSLSLSPGK*

Host Cells

Cells, including eukaryotic and prokaryotic cells, can be transformed with the expression vectors of the invention. Accordingly, another embodiment of the invention provides a host cell transformed with an expression vector of the instant invention. Cells of the invention are preferably eukaryotic cells, more preferably cells of plant, rodent, or human origin, for example but not limited to NSO, CHO, perC.6, Tk-ts13, BHK, or HEK293 cells.

Expression of the vectors of the instant invention in cells enables screening for cells having one or more phenotypes of interest, for example, cells exhibiting desired growth characteristics or proteins with desired phenotypes. Such desired phenotypes may include cells with enhanced growth rates, reduced requirements of nutrients or serum, reduced aggregates, growth at high density, reduced apoptosis, cells yielding high titers of recombinant protein, altered protein forms due to processing or cleavage, altered post translational moieties, and/or altered secondary folding isoforms, among other characteristics. Once a cell exhibiting a novel phenotype or phenotypes is derived, it can be expanded and selected, for example, for subclones that no longer express the recombinant nucleic acid sequence encoding the polypeptide of interest via selection. The derived cell is then suitable for expression of other proteins, antibodies, or antibody fragments.

“Transformed host cells” refers to cells into which the expression vectors of the instant invention have been introduced. Various cell culture systems can be employed to create transformed host cells; any cell line capable of expressing an appropriate vector may be used. Examples of suitable host mammalian cell lines include the COS-7 lines of monkey kidney cells, as described by Gluzman (1981) Cell 23:175; other suitable lines include HEK293 (Nicolaides et al. 1998), T98G, CV-1/EBNA, L cells (Holst et al. (1988)), C127, 3T3, Chinese hamster ovary (CHO) (Weidle, et al. (1988)), HeLa, TK-ts13 (Nicolaides et al. (1998)), NS1, Sp2/0 myeloma cells, and BHK cell lines, among others.

In general, transfection will be carried out using a suspension of cells, or a single cell, although other methods can also be applied to the extent that sufficient fraction of the treated cells or tissue incorporates the polynucleotide, thereby allowing transfected cells to be grown and utilized. Techniques for transfection are well known. Several transformation protocols are known in the art. See, e.g., Kaufman (1988) Meth. Enzymology 185:537. As is readily understood by those skilled in the art, the appropriate transformation protocol is determined by the host cell type and the nature of the gene of interest. The basic components of any such protocol include introducing nucleic acid sequence encoding the protein of interest into a suitable host cell, and then identifying and isolating host cells which have incorporated the vector DNA in a stable, expressible manner. Techniques for introducing polynucleotides include but are not limited to electroporation, transduction, cell fusion, the use of calcium chloride, and packaging of the polynucleotide together with lipid for fusion with the cells of interest. If the transfection is stable, such that the selectable marker gene is expressed at a consistent level for multiple cell generations, then a cell line results.

One common method for transfection into mammalian cells in particular is calcium phosphate precipitation as described in Nicolaides et al. (1998). Another method is polyethylene glycol (PEG)-induced fusion of bacterial protoplasts with mammalian cells. Schaffner et al. (1980) Proc. Natl. Acad. Sci. USA 77:2163. Yet another method is electroporation, which can also be used to introduce DNA directly into the cytoplasm of a host cell, as described, for example, in Potter et al. (1988) Proc. Natl. Acad. Sci. USA 81:7161.

Transfection of DNA can also be carried out using polyliposome reagents such as Lipofectin and Lipofectamine (available from Gibco BRL, Gaithersburg, Md.) which form lipid-nucleic acid complexes (or liposomes), which, when applied to cultured cells, facilitate uptake of the nucleic acid into the cells.

Once a cell is transfected, pools are selected to identify cells that have taken up the expression vector. Useful dominant selectable markers include antibiotic resistance genes, such as but not limited to, those conferring resistance to neomycin, kanamycin, tetracycline, hygromycin, or penicillin.

Transfected cells may be selected in a number of ways. For cells in which the vector also contains an antibiotic resistance gene, the cells may be selected for antibiotic resistance, which positively selects for cells containing the vector. In other embodiments, the cells may be allowed to grow under selective conditions, or may be further treated with a mutagen to enhance the rate of mutation and selected based on, for example, the presence of altered phenotypic characteristics of a gene or genes of interest, or according to a cell line characteristic. Once a phenotype of interest is achieved, the cells may be negatively selected based on the negative selection gene such that a null cell is obtained. As used throughout the instant disclosure, the term “null cell” refers to a cell or population of cells that no longer expresses a formerly-introduced recombinant protein. The loss of expression may be due to complete loss of the recombinant vector or through partial deletion of the vector such that the recombinant protein is no longer produced.

Once a clone producing a protein is identified, the line can be further screened to identify subclones having one or more desired phenotypes, such as but not limited to cells that exhibit high-titer expression, enhanced growth properties, and/or the ability to yield proteins with desired biochemical characteristics, for example, due to protein modification and/or altered post-translational modifications.

These phenotypes may be due to inherent properties of a given subclone or to mutagenesis. Mutagenesis can be effected through the use of chemicals, UV-wavelength light, radiation, viruses, insertional mutagens, defective DNA repair, or a combination of such methods.

Once a cell line that yields antibodies, antibody fragments, or polypeptides with one or more desired features is identified, the cell line can be subjected to selection conditions to identify clones that no longer contain the nucleic acid molecule containing the polypeptide of interest. For example, cells containing the HSV-TK selection marker can be treated with ganciclovir (GCV), a prodrug that is converted into a toxic nucleoside analog in cells expressing the HSV-TK gene (Carrio et al. (2001) Int. J. Cancer 94:81-88). Because the polypeptide of interest and HSV-TK are produced from the same transcript, clones that survive GCV treatment do not express the fusion transcript and are null for the polypeptide of interest. The null cell can be used to produce other proteins, antibodies or antibody fragments. The null cell preferably has the desired phenotype, e.g., high-titer expression, enhanced growth properties, and/or the ability to yield proteins with desired biochemical characteristics, for example, due to protein modification and/or altered post-translational modifications.

The use of fusion transcripts encoding a polypeptide of interest and a selection marker has advantages for recombinant methods employing recombinant expression vectors to screen for cells producing secreted polypeptides, nonsecreted polypeptides, and/or antibodies for use in production of other protein or antibody products.

In another embodiment, a method is provided for identifying a cell null for a nucleic acid sequence encoding the recombinant polypeptide. A vector encoding a polypeptide or antibody is introduced into a target cell and the cell line is selected for uptake of the vector by positive selection. Pools are generated and selected for subclones expressing the polypeptide of interest and exhibiting one or more desired phenotypes. Upon selection of desired subclones, these subclones are expanded and then negatively selected for further subsets that no longer express the recombinant polypeptide or antibody (“null”). The null cell preferably has a desired phenotype.

The invention provides methods for obtaining a cell line that displays the phenotype for production of recombinant proteins, antibodies, or antibody fragments. The method includes transforming a host cell with an expression vector of the instant invention, culturing the transformed host cell under conditions promoting novel expression or growth characteristics of the cell line, and selecting or screening for subclones exhibiting new phenotypes. In a preferred application of this method, transformed host cell lines are screened with two selection steps, the first to select or screen for cells with a new phenotype, and the second for negative selection of the selection marker sequence, in order to isolate subclones of the cell line that express the desired phenotype but no longer express or contain the recombinant protein or antibody vector. In a most preferred embodiment, the negative selection agent is ganciclovir, a prodrug that has been shown to cause toxicity to cells expressing the HSV-TK gene.

In some embodiments, cells containing an expression vector encoding a desired protein or antibody, such as the pIRES-pro-TK or pIRES-MAB-TK vectors, may be cultured with a mutagen to increase the frequency of genetic mutations in the cells. The mutagen may be withdrawn upon identification and selection or screening of cells displaying a desired altered phenotype, and either positive or negative selection may be performed, depending on whether the desired effect is to retain or remove cell lines that contain the original expression vector. In a further embodiment, a nucleic acid sequence encoding a new polypeptide, antibody, and/or antibody fragment of interest is introduced into the null cell.

The following references, each of which are incorporated herein by reference in their entirety, may be consulted for additional information on the relevant background technology:
1) Nicolaides, N. C. et al. (1998) Mol. Cell. Biol. 18:1635-1641.
2) Nicolaides, N. C. et al. (1997) Proc. Natl. Acad. Sci. USA 94:13175-13180.
3) Grasso, L. et al. (1998) J. Biol. Chem. 273:24016-24024.
4) Grosveld, F. et al. (1987) Cell 51:975-985.
5) Holst, A. et al. (1988) Cell 52:355-365.
6) Kaufman, R. et al. (1991) Nucl. Acids Res. 19(16):4485-4490, 1991.
7) Klehr, D. et al. (1991) Biochemistry 30:1264-1270.
8) McBratney, S. et al. (1993) Curr. Opin. Cell Biol. 5:961-965.
9) Wegner, M. et al. (1990) J. Biol. Chem. 265(23):13925-13932.
10) Weidle, U. et al. (1988) Gene 66:193-203.
11) Chen, L. et al. (2004) J Immunol Methods 295:49-56.
12) Yoon, S K. et al. (2004) Biotechnol Prog. 20:1683-1688.
13) Bohm, E. et al. (2004) Biotechnol Bioeng. 88:699-706.
14) Grasso, L. et al. (2004) Bioprocessing Intl. 2:58-64.

The above disclosure generally describes certain aspects of the present invention. Additional information thereon may be acquired by reference to the following specific examples, which are provided herein for purposes of illustration only, and are not intended to limit the scope of the invention.

### EXAMPLES

### Example 1

### Engineering the Recombinant Protein/Negative Selection Fusion

To demonstrate the functionality of recombinant protein/selection marker fusions to screen for a host cell with desired characteristics of growth and/or production, the pIRES-pro-TK and pIRES-MAB-TK vectors were each constructed. The pCMV vector containing the CMV promoter, followed downstream by a multiple polylinker cloning site and an SV40 polyadenylation signal, was used as a backbone along with a constitutively expressed neomycin phosphotransferase gene as a dominant positive selectable marker. The pCMV cassette contained an internal ribosome entry site (IRES) from the encephalomyocarditis virus (ECMV) that was cloned within the polylinker region. The recombinant gene encoding the Factor IX cDNA or other cDNAs was cloned into the EcoRI-XbaI site located upstream of the IRES sequence. A modified HSV-TK gene (SEQ ID: NO 1) was inserted downstream of the IRES sequence. To provide antibody production, the recombinant antibody sequence pIRES-MAB-TK was made by introducing a full length light chain cDNA (SEQ ID NO: 4) and a full length heavy chain cDNA (SEQ ID NO: 6) separated by an IRES into the recombinant gene cloning region. This vector has also yielded expression of other antibody genes. FIG. 1 provides schematics that depict pIRES-pro-TK (FIG. 1A) and pIRES-MAB-TK (FIG. 1B).

### Example 2

### Generation of Cells Expressing Protein from Recombinant Gene/Negative Selection Fusion Vectors

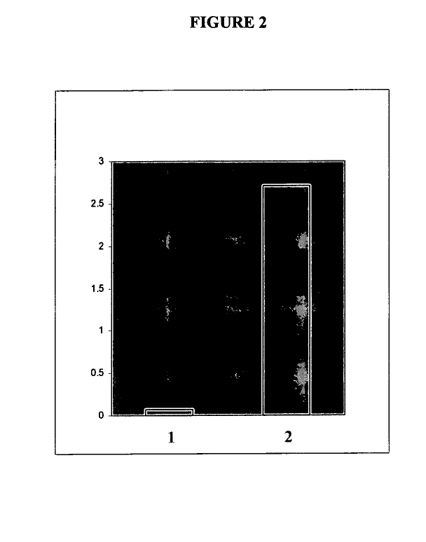

To demonstrate the ability to use a recombinant protein expression vector fused to a negative selection marker as a means of selecting cells exhibiting enhanced phenotypes, the pIRES-MAB-TK plasmid containing a full-length antibody was transfected into a cell line. In one embodiment, mammalian cells were transfected by electroporation according to the manufacturer's specifications. Transfected pools were selected for 10-14 days in 0.4 mg/ml of G418 (neomycin analog) to select for clones containing the expression vector. Next, cells were analyzed for gene expression via western blot or ELISA monitoring for recombinant protein (immunoglobulin, referred herein as Ig) expression. For western blot, 50,000 cells from the pIRES-MAB-TK culture or controls were centrifuged, and then resuspended in 150 microliters of 2×SDS buffer. Cultures were then analyzed for antibody expression by western blot. Western blots were carried out as follows: 50 microliters of pIRES-MAB-TK-transfected or empty vector culture were directly lysed in 2×lysis buffer (60 mM Tris, pH 6.8, 2% SDS, 10% glycerol, 0.1 M 2-mercaptoethanol, 0.001% bromophenol blue) and samples were boiled for 5 minutes. Lysate proteins were separated by electrophoresis on 4-20% Tris glycine gels (Novex). Gels were electroblotted onto Immobilon-P (Millipore) in 48 mM Tris base, 40 mM glycine, 0.0375% SDS, 20% methanol and blocked overnight at 4° C. in Tris-buffered saline plus 0.05% Tween-20 and 5% dry milk. Filters were probed with a monoclonal mouse antibody generated against the human immunoglobulin (Ig), followed by a secondary goat anti-mouse horseradish peroxidase-conjugated antibody. After incubation with the secondary antibody, blots were developed using chemiluminescence (Pierce) and exposed to film to measure Ig expression. These data were confirmed by ELISA monitoring for secretion of human Ig from transfected (FIG. 2, lane 2) and parental cells (FIG. 2, lane 1). ELISAs were carried out following the methods of Grasso et.al. (2004). Briefly, cells were plated in 96-well plates and grown for 24-76 hours. Aliquots of supernatants were isolated and incubated in 96-well test plates. Supernatants were quantified by ELISA using a mouse anti-human Ig antibody followed and HRP-conjugated anti-mouse antibody for detection. Ig content was determined as a function of substrate conversion and was captured by spectrophotometry.

ELISA assays of parental cells or cells transfected with pIRES-MAB-TK demonstrated the ability of pIRES-MAB-TK-transfected cells to generate robust antibody levels in Chinese Hamster Ovary Cells. FIG. 2 shows a representative value of Ig by ELISA from parental (lane 1) or pIRES-MAB-TK transfected (lane 2) CHO cells. The data is given in OD units.

### Example 3

### Generation of Negatively Selected Subclones from Recombinant Gene(s)/Selection Marker Fusion Vectors

Selected clones expressing Ig, as described in Example 2, are expanded and grown as single cell clones to identify desired subclones, for example, those that express high-titers or that exhibit preferred growth profiles. Once cells that exhibit a desired phenotype are identified, the subclones are expanded and then negatively selected for cells that are null for the expression vector (e.g., for pIRES-pro-TK or pIRES-MAB-TK), yielding clones with enhanced phenotypes that may be used to produce other proteins or antibodies from the optimized cell host. In order to obtain populations of null cells that exhibit a desired phenotype, one may wish to remove or completely suppress the expression of the original recombinant gene. For example, pIRES-MAB-TK cultures are grown for 5 days in the presence of the prodrug ganciclovir (Sigma), which kills cells expressing the HSV-TK gene product. After 5 days of negative selection, cells are grown for an additional 10 days in growth media alone at which time greater than 95% of cells die off. Resistant clones are then picked and expanded in 10 cm petri dishes. Cells are grown for 3 weeks, after which time a portion is reanalyzed for recombinant protein expression, for example, by western blot, RT-PCR, and/or PCR. DNA is analyzed for the presence of the pIRES-pro-TK or pIRES-MAB-TK vector by PCR using any set of primers that can specifically detect the presence or absence of the pIRES vector, for example, according to methods as previously described in Grasso et al. (1998).

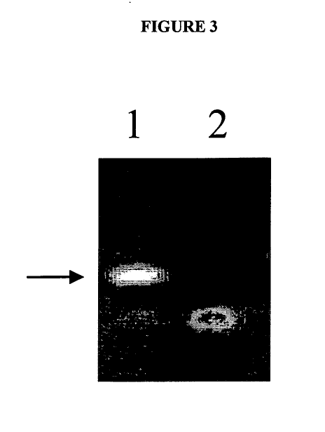

FIG. 3 demonstrates a typical result observed in the ganciclovir-resistant cells. Analysis of negatively selected clones exhibited the loss of the pIRES-pro-TK vector (FIG. 3, lane 2), while the presence of the vector was observed in the untreated cultures (as shown by an arrow in FIG. 3, lane 1). FIG. 3 depicts analysis of parental cells or cells transfected with pIRES-pro-TK, showing the ability to generate null cells. Shown is a representative evaluation of DNA from CHO cells that contain a pIRES-pro-TK vector, before and after negative selection by ganciclovir.

Further genetic analysis of the null subclones has demonstrated that, upon negative selection, resultant clones typically lose the entire vector sequence, thereby making the cells re-sensitive to neomycin selection (data not shown). These cells would now be suitable for introduction of any type of expression vector for production of recombinant proteins or antibodies or derivatives thereof. The expression vector for the to be introduced following loss of the expression vector of the invention may possess the structure shown for pIRES-pro-TK or pIRES-MAB-TK, or may possess only some of the components of those vectors, or may be entirely different.

The disclosures of each patent, patent application and publication cited or described in this document are hereby incorporated herein by reference, in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A polynucleotide vector comprising at least one nucleic acid sequence encoding a recombinant protein and at least nucleic acid sequence encoding a selection marker.

2. The vector of claim 1 wherein nucleic acid sequence encoding the selection marker is upstream of the nucleic acid sequence encoding the recombinant protein.

3. The vector of claim 1 wherein the nucleic acid sequence encoding the recombinant protein is upstream of the nucleic acid sequence encoding the selection marker.

4. The vector of claim 1 further comprising one or more internal ribosome entry sites.

5. The vector of claim 4 wherein the internal ribosome entry site is positioned between the nucleic acid sequence encoding the recombinant protein and the nucleic acid sequence encoding the selection marker.

6. The vector of claim 1 comprising one or more promoters operatively linked to at least one of said nucleic acid sequences.

7. The vector of claim 1 wherein the selection marker is a negative selection marker.

8. The vector of claim 7 wherein the negative selection marker is herpes simplex virus thymidine kinase (HSV-TK) or a derivative thereof.

9. The vector of claim 8 wherein the negative selection marker is encoded by a nucleic acid sequence of SEQ ID NO: 1.

10. The vector of claim 1 further comprising a nucleic acid sequence encoding is a positive selection marker.

11. The vector of claim 1 further comprising at least one polyadenylation signal.

12. The vector of claim 11 wherein the polyadenylation signal is downstream of the nucleic acid sequence encoding the selection marker.

13. The vector of claim 11 wherein the polyadenylation signal is upstream of the nucleic acid sequence encoding the selection marker.

14. The vector of claim 11 wherein the polyadenylation signal is downstream of the nucleic acid sequence encoding the recombinant protein.

15. The vector of claim 11 wherein the polyadenylation signal is upstream of the nucleic acid sequence encoding the recombinant protein.

16. The vector of claim 6 wherein the promoter is a constitutive promoter, inducible promoter, tissue-specific promoter, or host-specific promoter.

17. A cell comprising a vector according to claim 1.

18. The cell according to claim 17, wherein said cell is a eukaryotic cell.

19. The cell according to claim 18, wherein said cell is a mammalian cell.

20. A method for producing an isolated, genetically stable cell with a desired phenotype comprising the steps of:
a) culturing a cell under conditions for the expression of a recombinant polypeptide thereby producing a cell library;
b) selecting clones from the cell library that exhibit new phenotypes;
c) expanding the selected clones; and,
c) selecting clones no longer expressing the recombinant polypeptide.

21. The method of claim 20 wherein said cell comprises a vector comprising one or more nucleic acid sequences encoding the recombinant polypeptide and one or more nucleic acid sequences encoding a selection marker.

22. The method of claim 20 wherein said cell comprises a vector comprising:
a promoter or an internal ribosome entry site, operatively linked to at least one nucleic acid sequence encoding an immunoglobulin light chain;
a nucleic acid sequence encoding an immunoglobulin heavy chain, separated from the nucleic acid sequence encoding the immunoglobulin light chain by at least one internal ribosome entry site; and,
at least one selection marker sequence preceded upstream by an internal ribosome entry site or promoter.

23. The method of claim 20 wherein said cell comprises a vector comprising:
a promoter or an internal ribosome entry site, operatively linked to at least one nucleic acid sequence encoding an immunoglobulin heavy chain;
a nucleic acid sequence encoding an immunoglobulin light chain, separated from the nucleic acid sequence encoding the immunoglobulin heavy chain by at least one internal ribosome entry site; and,
at least one selection marker sequence preceded upstream by an internal ribosome entry site or promoter.

24. The method of claim 21 wherein said selection marker is a negative selection marker.

25. The method of claim 21 wherein said vector further comprises a positive selection marker.

26. The method of claim 21 wherein said vector comprises one or more promoters operatively linked to at least one of said nucleic acid sequences.

27. The method of claim 21 wherein the nucleic acid sequence encoding the selection marker is upstream of the nucleic acid sequence encoding the recombinant polypeptide.

28. The method of claim 21 wherein the nucleic acid sequence encoding the recombinant polypeptide is upstream of the nucleic acid sequence encoding the selection marker.

29. The method of claim 21 wherein the vector comprises one or more internal ribosome entry sites.

30. The method of claim 29 wherein the internal ribosome entry site is positioned between the nucleic acid sequence encoding the recombinant polypeptide and the nucleic acid sequence encoding the selection marker.

31. The method of claim 23 wherein the negative selection marker is herpes simplex virus thymidine kinase (HSV-TK) or a derivative thereof.

32. The method of claim 31 wherein the negative selection marker is encoded by a nucleic acid sequence of SEQ ID NO: 1.

33. The method of claim 21 wherein the vector comprises at least one polyadenylation signal.

34. The method of claim 33 wherein the polyadenylation signal is downstream of the nucleic acid sequence encoding the selection marker.

35. The method of claim 33 wherein the polyadenylation signal is upstream of the nucleic acid sequence encoding the selection marker.

36. The method of claim 33 wherein the polyadenylation signal is downstream of the nucleic acid sequence encoding the recombinant polypeptide.

37. The method of claim 33 wherein the polyadenylation signal is upstream of the nucleic acid sequence encoding the recombinant polypeptide.

38. The method of claim 26 wherein the promoter is a constitutive promoter, inducible promoter, tissue-specific promoter, or host-specific promoter.

39. The method of claim 21 wherein the vector is pIRES-pro-TK.

40. The method of claim 21 wherein the vector is pIRES-MAB-TK.

41. The method of claim 20 wherein said host cell is a mammalian cell.

42. The method of claim 20 wherein said host cell is a plant cell.

43. The method of claim 20 wherein said host cell is an amphibian cell.

44. The method of claim 20 wherein said host cell is an insect cell.

45. The method of claim 20 wherein said host cell is a fungal cell.

46. The method of claim 20 further comprising inducing mutagenesis during said culturing step.

47. The method of claim 46 wherein said step of inducing mutagenesis comprises treating said cell with a mutagen during said culturing step.

48. The method of claim 46 wherein said step of inducing mutagenesis comprises inhibiting mismatch repair of the cell during said culturing step.

49. A cell produced according to the method of claim 20.

50. A vector comprising:
a promoter or an internal ribosome entry site, operatively linked to at least one nucleic acid sequence encoding an immunoglobulin light chain;
a nucleic acid sequence encoding an immunoglobulin heavy chain, separated from the nucleic acid sequence encoding the immunoglobulin light chain by at least one internal ribosome entry site; and,
at least one selection marker sequence preceded upstream by an internal ribosome entry site or promoter.

51. The vector according to claim 50 wherein the selection marker is a negative selection marker.

52. The vector according to claim 51 wherein the negative selection marker is herpes simplex virus thymidine kinase (HSV-TK) or a derivative thereof.

53. The vector of claim 52 wherein the nucleic acid sequence encoding the selection marker comprises the nucleotide sequence of SEQ ID NO: 1.

54. The vector of claim 50 wherein said immunoglobulin light chain comprises an amino acid sequence of SEQ ID NO:5.

55. The vector of claim 50 wherein said nucleic acid sequence encoding an immunoglobulin light chain comprises a nucleic acid sequence of SEQ ID NO:4.

56. The vector of claim 50 wherein said immunoglobulin heavy chain comprises an amino acid sequence of SEQ ID NO:7.

57. The vector of claim 50 wherein said nucleic acid sequence encoding an immunoglobulin heavy chain comprises a nucleic acid sequence of SEQ ID NO:6.

58. The vector of claim 50 comprising at least one polyadenylation signal.

59. A cell comprising a vector according to claim 50.

60. The cell according to claim 59, wherein said cell is a eukaryotic cell.

61. A cell containing a recombinant expression vector comprising a promoter operatively linked to a first nucleic acid sequence, said first sequence encoding a recombinant cDNA or a selection marker sequence, and a second nucleic acid sequence, said second sequence encoding either a recombinant cDNA or a selection marker sequence.

62. A cell according to claim 61 wherein said first nucleic acid sequence encodes a recombinant light chain and heavy chain cDNA.

63. A cell according to claim 62 wherein said first nucleic acid sequence encodes a selection marker.

64. A cell according to claim 61 wherein said first nucleic acid sequence is separated from said second nucleic acid sequence by an internal ribosome entry site.

65. A cell according to claim 61 wherein the recombinant expression vector is pIRES-MAB-TK or pIRES-pro-TK.

66. The cell of claim 61 wherein said cell is eukaryotic.

67. The cell of claim 61 wherein said cell is mammalian.

68. The cell of claim 61 wherein said cell is prokaryotic.

69. A method for determining the presence of pIRES-pro-TK in a cell comprising the steps of:
a. transfecting cells with pIRES-pro-TK; and
b. analyzing the cells of step (a) using primers or DNA probes that can specifically detect said vector.

70. A method for determining the presence of pIRES-MAB-TK vector comprising the steps of:
a. transfecting cells with pIRES-MAB-TK; and
b. analyzing the cells of step (a) using primers or DNA probes that can specifically detect said vector.
